(19) United States
(12) Patent Application Publication
(10) Pub. No.: US 2024/0335330 A1
(43) Pub. Date: Oct. 10, 2024

(54) FLAT-KNIT COMPRESSION GARMENT

(71) Applicant: SIGVARIS AG, St. Gallen (CH)

(72) Inventors: Frank Bauer, Flawil (CH); Julia Sabrina Fleischer, St. Gallen (CH)

(21) Appl. No.: 18/579,315

(22) PCT Filed: Jun. 23, 2022
(86) PCT No.: PCT/EP2022/067095

§ 371 Date: Jun. 26, 2024

(30) Foreign Application Priority Data
Jul. 15, 2021 (CH) 070057/2021

(51) Int. Cl. A61F 13/08; D04B 1/10; D04B 1/18; D04B 1/24

(52) U.S. Cl. CPC A61F 13/08; D04B 1/108; D04B 1/18; D04B 1/24; D10B 2331/02; D10B 2331/10; D10B 2509/00

(57) ABSTRACT

The invention relates to a flatknitted compression garment comprising a knitted fabric with at least one indentation. The at least one indentation comprises a concave indentation (21) which is designed in such a way that, when used as intended, the knitted fabric forms an inwardly curved surface when viewed from a wearer of the compression garment. The concave indentation (21) is designed in particular such that the knitted fabric, when used as intended, lies against a concave body shape of the wearer and thus has a medical effectiveness, e.g. in the treatment of lipedema, lymphedema and/or phlebedema, while at the same time being very comfortable to wear.

### FIELD OF THE INVENTION

The invention relates to a flatknitted compression garment, in particular compression pants, and to their use in the treatment of lipedema and/or lymphedema.

### BACKGROUND

Compression garments are used as standard for the care and treatment of lipedema, a disease involving an increase in fatty tissue in the subcutaneous fat tissue, and lymphedema, an accumulation of fluid in the intercellular space, or a mixed form of several diseases such as lipedema, lymphedema and/or phlebedema. The compression garment may, for example, be a compression stocking or compression pants. If the compression garment is adapted to the body mass of the wearer—i.e. the person wearing the compression garment—it relieves the damaged tissue by compressing the enclosed part of the wearer's body. In this way, the compression garment can prevent lipedema, lymphedema and/or phlebedema from developing and growing.

To be medically effective, the compression garment must fit tightly against the body, i.e. there should be as few gaps as possible between the compression garment and the body. For the treatment of lipedema, lymphedema and/or phlebedema, flatknitted compression garments are primarily used as they have a higher material stiffness.

Medical compression stockings and arm garment may be certified in accordance with the RAL-GZ 387/1 and RAL-GZ 387/2 quality assurances, which define different compression classes. In particular, these are four compression classes with compression intensities of “1-light”, “2-medium”, “3-strong” and “4-very strong” and associated ranges of compression pressures of 18-21 mmHg or 15-21 mmHg, 23-32 mmHg, 36-42 mmHg and 49 mmHg and greater. The most common class is compression class 2.

The material stiffness (=Stiffness) describes the increase in pressure that results from 1 cm of additional stretching of the compression garment. Material stiffness is therefore inversely related to extensibility. In other words, material stiffness describes the ability of the compression garment to act as a firm abutment when the muscles contract, e.g. during movement. Compression garments with a high material stiffness are called “short-stretch”, those with a low material stiffness “long-stretch”.

Compression garments are known which have one or more bulges or curvatures and thus generate more volume for certain parts of the body. This can be achieved by an indentation for convex parts of the body. Such indentations are used, for example, on joints such as the heel, knee or elbow or on the buttocks. They are also called “elliptical indentations”. The aim of such indentations for a convex body shape is to improve mobility and/or to relieve strain when, for example, the foot or arm is permanently bent and/or to provide a butt-shaping body part.

Concave body shapes of the wearer are problematic in the treatment of lipedema, lymphedema and/or phlebedema. “Concave” means in particular that the surface of the body has a depression or constriction at this point, i.e. it is curved inwards when viewed from the body. On concave body shapes, the compression garment may not fit tightly against the body. This results in a cavity forming between the compression garment and the body. Lymphatic fluids and fat cells may accumulate in such a cavity, which may lead to edema.

For anatomical reasons, almost everyone has a concave shape, particularly in the groin. When wearing a compression garment, the cavities mentioned above can therefore occur between the knitted fabric and the body surface. This is particularly pronounced if the abdominal protrusion is low and the belly protrudes far forward. In known compression pants, an attempt is made to counteract the formation of cavities in the groin by running a horizontal seam in the groin area, see also FIG. 1 and the associated description in the section “Ways of carrying out the invention”. Such compression pants thus comprise two leg parts as well as a front and a rear body part. However, the seam running between the front body part and the leg part(s) in particular often leads to constrictions and rubs unpleasantly, so that wearing comfort is reduced.

The task is therefore to provide a medically effective compression garment that in particular avoids cavities between the compression garment and the body surface and at the same time is comfortable to wear.

### DISCLOSURE OF THE INVENTION

The task is solved by a flatknitted compression garment comprising a knitted fabric with at least one indentation for a concave body shape according to claim 1.

For the “presentation of the invention”, the definitions from the previous section are included.

In particular, “concave” is a surface that is curved inwards—or in other words “recessed”-when viewed from the wearer of the compression garment or from the body of the wearer. Mathematically defined, a body with a concave surface has no straight connecting line within the body between any two points located on the concave surface.

An “indentation” is an area in the knitted fabric in which the number of stitches per row changes. The number of stitches per row in the knitted fabric varies, i.e. it increases or decreases. In particular, at an indentation several stitches of a row are knitted together with a stitch of a subsequent row of stitches. In particular, the indentation comprises a decreasing number of stitches per row of stitches up to a reversal point and an increasing number of stitches from the reversal point onwards. The decrease or increase in the number of stitches may occur over several rows of stitches (continuously) or abruptly by changing the number by several stitches at the same time. This also leads to a three-dimensional structure, in other words a “curvature”, in the knitted fabric.

In a compression garment according to the claim, the compression garment comprises at least one concave indentation, i.e. an indentation for a concave body shape. It is designed in such a way that, when used as intended, the knitted fabric forms an inwardly curved surface when viewed from a wearer of the compression garment. In particular, this reduces a volume within the compression garment and the knitted fabric lies against a concave body shape of the wearer when used as intended.

The indentation for a concave body shape is also called a “triangular indentation” and results in an inward curvature—or in other words a “depression” in the knitted fabric or a “narrowing” of the knitted fabric. The effect of the concave indentation is to avoid cavities between the compression garment and the body surface while ensuring a high level of comfort.

Furthermore, a “flatknitted” fabric is produced on a flat knitting machine. The flatknitted fabric is manufactured on one or more needle beds. This means that the number of stitches in each row may increase or decrease.

Advantageously, the concave indentation in a first row of stitches has one stitch on each needle at a first end of the row of stitches, i.e. at a first end of the row of stitches, each needle is used for knitting. At the second end of the row, however, the number of stitches decreases. The concave indentation therefore advantageously comprises an unchanged number of stitches at a first end and a reduced number of stitches at a second end in a first row of stitches. This reduces the total number of stitches per row, while the first end is knitted through in every row.

In a second, in particular subsequent, row of stitches, stitches are advantageously formed on each needle so that all stitches are picked up again on the needle. The concave indentation therefore comprises an unchanged number of stitches at both the first and second end in a second row of stitches.

In a third, in particular subsequent, row of stitches, the number of stitches is advantageously reduced at both the first and second end of the row of stitches in the case of a concave indentation. Advantageously, the stitches are offset to one side from row to row.

In particular, the concave indentation therefore comprises a decreasing number of stitches per stitch row, over several stitch rows, up to a reversal. As described, this does not rule out the possibility that the number of stitches between successive stitch rows within the concave indentation may remain the same.

In general, there may be several of the first, second and/or third rows of stitches in succession.

The concave indentation is characterized by the fact that all stitches on the needle are picked up again in the second row of stitches. This contrasts in particular with a convex indentation, in which the number of stitches decreases or increases in each row of stitches at both the first and second end of the rows of stitches.

Advantageously, the concave indentation also comprises a number of stitches per stitch row that increases again from the reversal point over several stitch rows. This does not exclude the possibility that the number of stitches between successive rows of stitches within the concave indentation may also remain the same.

A compression garment according to the above embodiments always fits tightly against the wearer's body, even with concave body shapes-even if the wearer is moving. In this way, the compression garment avoids cavities between the compression garment and the wearer's body and thus prevents the (further) formation of edema.

The compression garment fits the wearer's body evenly thanks to a concave indentation, i.e. the increasing and decreasing number of stitches rather than a seam. In addition to an improved fit, this also makes the compression garment more comfortable to wear.

In one embodiment, the knitted fabric comprises a knitting thread and a weft thread inserted in at least every second row of stitches, in particular in every row of stitches. The knitting thread and the weft thread are elastic. The knitting thread therefore forms the basic framework into which the weft thread is inserted in every second or every stitch row. Because the knitting thread and the weft thread are elastic, the knitted fabric builds up a force when stretched, which leads to the desired compressive effect.

With regard to the material, it is advantageous that at least the knitting thread and optionally also the weft thread consist of a coated elastane core. The sheathing may be single or double. In particular, sheathing is also understood to mean wrapping and spinning.

With regard to the stitch type, it is advantageous that the knitting thread is knitted right-left. When producing the knitted fabric on two needle beds, this means in particular that the stitches of the knitting thread are knitted on only one needle bed. Alternatively, the knitting thread may also be knitted right-right.

In addition, the knitted fabric advantageously comprises a plating yarn, which may be a textured polyamide yarn. With regard to the stitch type, it is preferable that the plating yarn is knitted in a right-right direction. When manufacturing the knitted fabric on two needle beds, this means in particular that the stitches of the plating yarn are knitted on both needle beds. Right-right knitted fabrics exhibit the same structure on both sides and therefore look the same from both sides.

Together with the stitch yarn, the plating yarn forms the basic structure and thus creates a stable and homogeneous knitted fabric.

In order to customize the feel, look and stretch properties of the knitted fabric, the knitted fabric may optionally include other yarns, e.g. another elastic knitting thread or another plating yarn.

In one embodiment, the compression garment is a pair of compression pants. The knitted fabric comprises a left and right leg part, in particular a left and right leg part, a front body part and a rear body part. The single leg part and the front body part are knitted in one piece, i.e. in particular there is no seam between the leg part and the front body part. The concave indentation is knitted in a groin area of the compression pants, in particular at a transition between the leg part and the front body part. As a result, the knitted fabric lies against a groin of the wearer when used as intended.

This prevents the formation of a cavity between the compression pants and the wearer's body in the groin area—and thus (further) edema formation. It also ensures that the compression pants fit well and are very comfortable to wear, especially since friction points and constrictions are avoided in the groin area, which experience has shown to occur in the case of, for example, a seam in the groin area.

Optionally, the rear body part may be connected to the front body part and the leg part by a seam. This is a good option as seams cannot be completely avoided in flatknitted compression pants, but are not very disturbing at the transition to a wearer's buttocks.

It is advantageous that the concave indentation runs at an angle of between 100 degrees and 150 degrees, in particular between 110 degrees and 130 degrees, to a longitudinal direction of the leg part. Ideally, the concave shape created by the indentation follows the groin of the wearer such that cavities between the compression pants and the wearer's body are avoided. For this purpose, it is advantageous that the shape of the indentation is adapted to the shape of the wearer's groin. In particular, the position and angle of the indentation are therefore individually designed and knitted according to the measures of a particular wearer. This achieves an optimum medical effect against lipedema, lymphedema and/or phlebedema.

In further embodiments, the concave indentation is used on other body shapes such as the hollow of the knee, instep and/or crook of the elbow. This achieves the same effects, in particular that the knitted fabric follows the shape of the body and, for example, fits tightly to the body even when moving and retains its compressive effect. The above-mentioned advantageous embodiments may also be applied to a concave indentation on the other body shapes.

A compression garment with a concave indentation is therefore particularly suitable for use in the treatment of lipedema, lymphedema and/or phlebedema. Advantageously, the knitted fabric is also short-stretch, see definition in the “Background” section, so that a better medical effect is achieved. In embodiments of the invention, this is achieved by manufacturing the knitted fabric as a flat knit.

### BRIEF DESCRIPTION OF THE DRAWINGS

Further embodiments, advantages and applications of the invention arise from the dependent claims and from the following description based the figures. Showing:

FIG. 1 a schematic side view of a part of a of compression pant from the prior art;

FIG. 2 a schematic side view of a part of a compression pants according to an embodiment of the invention;

FIG. 3 a diagram of a knitting construction for an indentation for a concave body shape (“triangular indentation”) according to an embodiment;

FIGS. 4a and 4b schematic knitting programs of a left respectively right front body part and leg part, each with a triangular indentation in the groin area according to an embodiment;

FIGS. 5a and 5b schematic knitting patterns of a left respectively right front body part and leg part, each with a triangular indentation in the groin area according to an embodiment;

FIG. 6 a microscope image of a knitted fabric with a triangular indentation according to an embodiment.

### WAYS TO CARRY OUT THE INVENTION

### Compression Pants

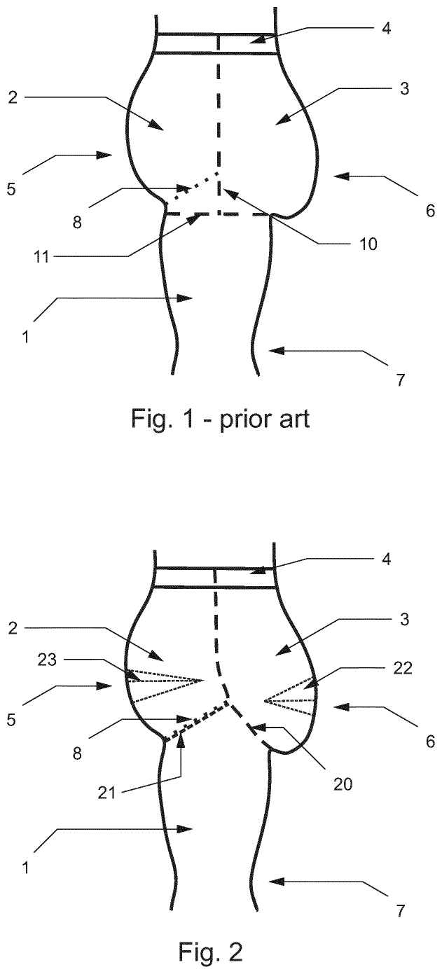

FIG. 1 shows compression pants with seams 10 and 11 as known in the prior art. The side view schematically shows: the belly 5, the buttocks 6, the hollow of the knee 7 and the groin 8 of the wearer of the compression pants. The compression pants comprise a leg part 1, a front body part 2 and a rear body part 3. The upper end of the compression pants in the intended use is formed by the waistband 4 on the front body part 2 and on the rear body part 3.

The general aim of medical compression pants is to support the wearer's body and thus relieve the eventually already damaged body tissue. In particular, compression is intended to prevent the further formation of edema, e.g. lipedema, lymphedema or phlebedema. To this end, the compression pants must fit tightly around the wearer's body.

In the compression pants from the prior art shown in FIG. 1, a good fit should be achieved by the division into leg part 1, front body part 2 and rear body part 3, wherein the front body part 2 and the rear body part 3 are connected with a seam 10 and the leg part 1 is attached to the body parts 2 and 3 with a further seam 11.

However, the horizontally extending seam 11 in particular often leads to constrictions, especially when the wearer moves, and to unpleasant friction points. In addition, the shown compression pants with seam 11 do not achieve a good fit in the groin 8. The formation of cavities in the concave body shape of the groin 8 is therefore not completely avoided.

FIG. 2 shows compression pants with a triangular indentation 21 according to an embodiment of the invention. Again, a schematic side view is shown with the belly 5, buttocks 6, hollow of the knee 7 and groin 8 of the wearer of the compression pants. Also these compression pants comprise a leg part 1, a front body part 2 and a rear body part 3. The rear body part 3 is again joined to the leg part 1 and the front body part 2 by a seam 20. However, the leg part 1 and the front body part 2 are knitted in one piece here. For a better fit and a better medical effect, however, there is a triangular indentation 21 at the groin 8. This means that the compression pants are curved inwards in the groin 8, i.e. they follow the concave body shape of the groin 8, such that there are no cavities and the desired compressive effect is also achieved in the groin area.

The triangular indentation 21 in compression pants is also called “triangular indentation”. This name comes from the geometric shape of the body at the abdominal protrusion or groin 8. In the side view of FIG. 2 it becomes clear that three lines meet at this point: the groin 8 respectively the indentation for concave body shape 21 (from top right), the silhouette of the belly 5 (from top left) and the silhouette of the thigh (from bottom). These three lines enclose with each other essentially equal angles, i.e. respectively approx. 120 degrees. “Essentially” includes, for example, a distribution of the angles 140 degrees/110 degrees/110 degrees. The triangular indentation or indentation for concave body shape 21 is therefore matched to the wearer's body shape, which leads to a good fit and medical effectiveness.

Optionally, the compression pants may also comprise one or more convex indentations 22 and 23. A convex indentation leads to an outwards curvature of the compression pants, i.e. away from the body, even in the unstretched state, e.g. without wearer. A convex indentation leaves more room for the wearer's body, especially in the case of convex body shapes. This leads to an even distribution of pressure and avoids painful constrictions. A convex indentation 22 in the area of the buttocks 6 and/or a convex indentation 23 in the area of the belly 5 is advantageous.

Both the indentation for a concave body shape 21 and the optional convex indentations 22 and 23 are ideally matched to the wearer. In a first step the wearer's body is measured. This includes taking the position and orientation of the groin as well as a measurement for the belly and, if necessary, a measurement for the buttocks. Based on the measurements, suitable compression pants are then selected or specifically designed for the wearer. In the latter case, the shape of the triangular indentation is created depending on the body measurements. Depending on the measurements, one or more indentations may be incorporated.

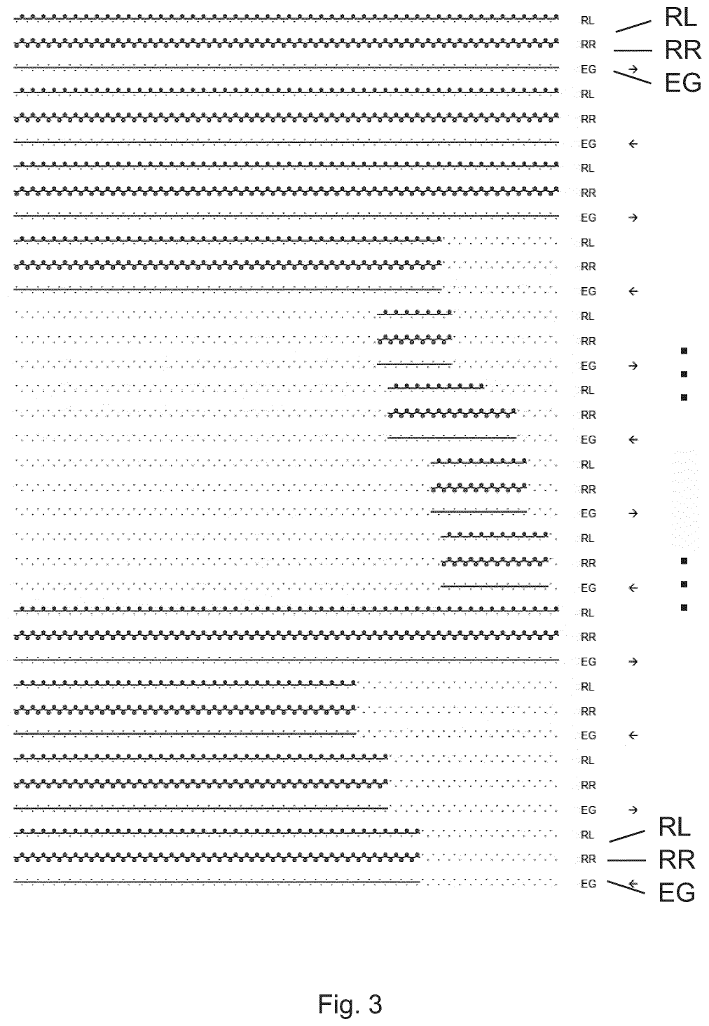

Triangular indentations or indentations for a concave body shape are not limited to use in compression pants, but may also be used in other compression garments, namely wherever a good fit and/or uniform compression is required for concave body shapes. The knitting construction and knitting pattern of a triangular indentation (FIG. 3) are generally described schematically below.

### Triangular Indentation

FIG. 3 shows a schematic of a knitting construction for a triangular indentation or indentation for a concave body shape. However, FIG. 3 shows a simplified knitting construction with a reduced number of stitch rows and stitches to illustrate the basic structure of the concave indentation. The side length of a real indentation would be approx. three times as long, depending on the body dimensions.

In the illustration in FIG. 3, RL, RR and EG are repeated again and again, namely the right-left (RL) stitches of the knitting yarn (also called elastic stitch yarn), the right-right (RR) stitches of the plating yarn and the inserted weft yarn (also called interlining yarn EG). A sequence of RL-RR-EG forms a stitch repeat in the knitted fabric.

For construction of a triangular indentation, the number of stitch bars or stitches per row is changed, e.g. as shown. The number of stitches decreases up to the reversal point—in FIG. 3 between the first EC and the second RL from the top—and then increases again. When knitting, the stitches that have no adjacent stitches in the next row up in FIG. 3 are joined to the stitches in the top row of stitches (RL, RR, EG). This creates a three-dimensional shape in the knitted fabric, namely a curvature—or in other words a depression or narrowing of the knitted fabric.

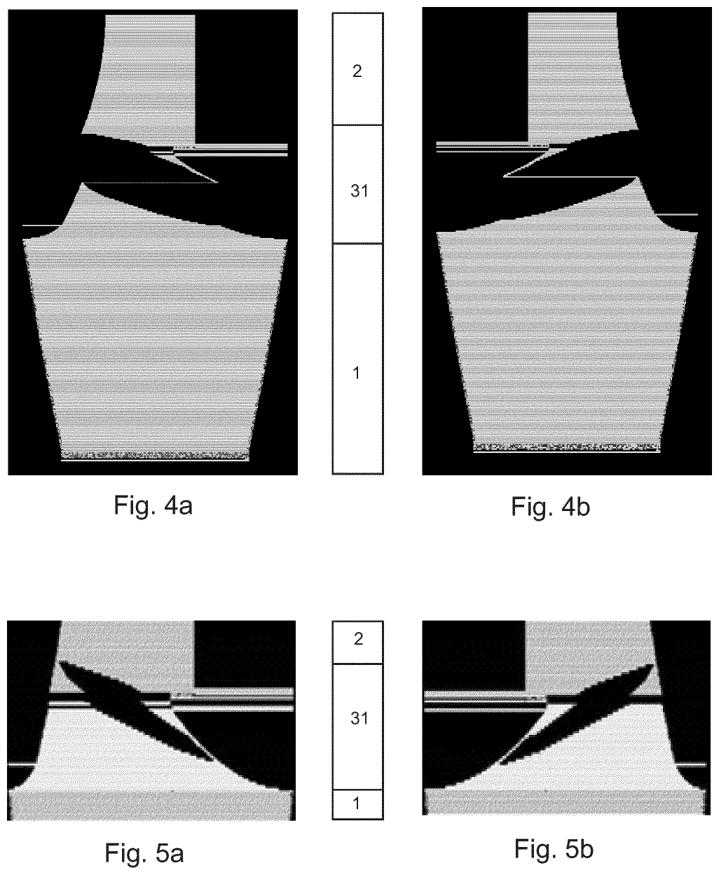

FIGS. 4a and 4b show schematic knitting programs of a left respectively right front body part 2 (upper area) and a leg part 1 (lower area), each with area 31 for a triangular indentation or for an indentation for a concave body shape in the groin. The knitting programs show how the knitting carriage moves, namely one row of stitches from left to right, the next row of stitches from right to left, etc. The continuous decrease in the number of stitches per row up to the reversal point and then the continuous increase can be clearly seen in area 31 of the triangular indentation.

FIGS. 5a and 5b show knitting patterns of a left respectively right front body part 2 and leg part 1, each with area 31 of the triangular indentation for the groin. The knitting patterns may be calculated from the knitting programs. In particular, the knitting patterns in FIGS. 5a and 5b are calculated on the basis of a section of the knitting programs in FIGS. 4a and 4b. The “hole” (black area) in the knitting pattern is clearly recognizable in the area 31 of the triangular indentation. However, there is no actual hole at this point in the finished knitted fabric, as the stitches adjacent to the “hole” at the bottom are knitted together with the adjacent stitches at the top. As a result, the knitted fabric is pulled together in the area 31 of the gusset, which leads to a narrowing or curvature, particularly inwards.

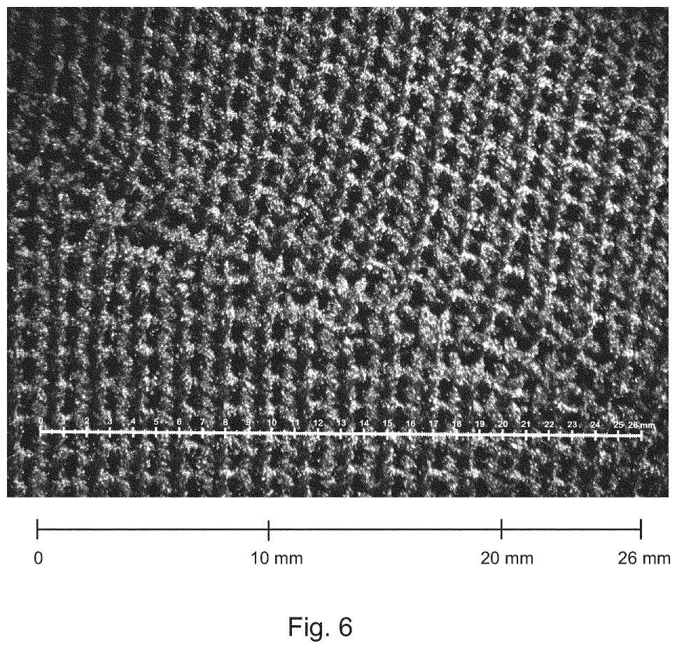

FIG. 6 shows a section of a knitted fabric with an indentation in a microscopic image. In reality, the section has a width of approx. 3 cm. The indentation runs from the left edge of the section, approximately one third below the top left corner, to the right edge, just above the bottom right corner. The effect of the changing number of stitches per row is clearly visible, which leads to a narrowing or curvature of the knitted fabric, in particular towards the inside.

While preferred embodiments of the invention are described in the present application, it should be clearly noted that the invention is not limited thereto and may be practiced in other ways within the scope of the following claims.

1. Flatknitted compression garment comprising a knitted fabric defining at least one indentation,
wherein the at least one indentation is a concave indentation, and
wherein, in the intended use, the knitted fabric forms an inwardly curved surface at the indentation when viewed from a wearer of the compression garment.

2. Compression garment according to claim 1,
wherein the knitted fabric, in the intended use, lies against a concave body shape of the wearer.

3. Compression garment according to one of claim 1 or 2,
wherein the knitted fabric forming the concave indentation in a first row of stitches comprises an unchanged number of stitches at a first end and a reduced number of stitches at a second end,
wherein the knitted fabric forming the concave indentation in a second row of stitches comprises an unchanged number of stitches at both the first and second ends,
in particular wherein the knitted fabric forming the concave indentation in a third row of stitches comprises a reduced number of stitches at both the first and second ends,
in particular wherein the knitted fabric forming the concave indentation comprises, up to a reversal point, a decreasing number of stitches per row of stitches over several rows of stitches.

4. Compression garment according to claim 3,
wherein the knitted fabric forming the concave indentation comprises a number of stitches per row of stitches which increases again from the reversal point over several rows of stitches.

5. Compression garment according to claim 4,
wherein the knitted fabric comprises a knitting thread and a weft thread inserted in at least every second row of stitches,
wherein the knitting thread and the weft thread are elastic,
and
wherein at least the knitting thread and the weft thread comprise at least a single coated elastane core.

6. Compression garment according to claim 5,
wherein the knitted fabric comprises a plating yarn,
and wherein the plating yarn is a texturized polyamide yarn.

7. Compression garment according to claim 1,
wherein the compression garment is a compression pants,
wherein the knitted fabric comprises a leg part, a front body part and a rear body part,
wherein the leg part and the front body part are knitted in one piece without a seam running between them,
wherein the concave indentation is knitted in a groin area of the compression pants at a transition between the leg part and the front body part, such that the knitted fabric lies against a groin of the wearer in the intended use, and
wherein the rear body part is connected to the front body part and the leg part by a seam.

8. Compression garment according to claim 7,
wherein the concave indentation extends at an angle of between 100 degrees and 150 degrees.

9. Compression garment according to one of claim 7 or 8,
wherein the shape of the indentation is adapted to the shape of the groin of the wearer.

10. Compression garment according to claim 1, wherein the compression garment is for use in the treatment of medical conditions such as lipedema, lymphedema and phlebedema.

11. Compression garment according to claim 5, wherein the knitting thread is knitted right-left.

12. Compression garment according to claim 7,
wherein the concave indentation extends at an angle of between 110 degrees and 130 degrees.
